(19) United States
(12) Patent Application Publication
(10) Pub. No.: US 2025/0229060 A1
(43) Pub. Date: Jul. 17, 2025

(54) Retainer System for Catheter

(71) Applicant: C. R. Bard, Inc., Franklin Lakes, NJ (US)

(72) Inventors: Surender Gunasekar, Ranipet (IN); Paramanandan Balasubramaniam, Chennai (IN)

(21) Appl. No.: 18/414,408

(22) Filed: Jan. 16, 2024

(51) Int. Cl. A61M 25/02; A61M 25/00

(52) U.S. Cl. CPC A61M 25/02; A61M 25/0017; A61M 25/0021; A61M 2025/028; A61M 2202/0014; A61M 2202/0496; A61M 2209/088; A61M 2210/04; A61M 2210/1085

(57) ABSTRACT

A retainer system includes a catheter tube, a bifurcation, a drainage extension leg and an inflation extension leg. The system can also include an anchor pad and a retainer rotatably coupled to a top surface of the anchor pad. The retainer can include a base and a cap hingedly coupled to the base. Additionally, the retainer can feature one or more extension leg sleeves with a lumen, extending from the first side of the retainer, and a catheter tube sleeve with a lumen, extending from the second side of the retainer. The sleeve can define a serpentine shape to impart a predetermined amount of slack on the catheter and mitigate dislodgement of the catheter distal tip when the retainer system moves relative thereto. Further, the retainer system can mitigate kinking or collapse of the catheter by providing strain relief between the catheter and the retainer system.

### BACKGROUND

When placing a drainage catheter, a distal tip is disposed within a cavity of the body and a proximal end is disposed external to the body and is in fluid communication with a collection container. Often the proximal end is releasably secure to a skin surface of the patient by a retainer system. Exemplary retainer systems often include an anchor pad that adheres to a skin surface of the patient and a retainer device coupled thereto for securing proximal portion in place. Care must be taken in the positioning of the retainer system to ensure there is sufficient slack in the catheter tube so as not to pull on the catheter during everyday movement or use. Too little slack may cause discomfort to the patient and/or cause the catheter to disconnect from the collection container.

Alternatively, care must be taken to ensure that is not too much slack which may cause the formation of dependent loops that cause pooling of the fluid within the catheter, retrograde fluid flow, or the catheter may get twisted or tangled on itself, causing kinking or collapse of the catheter, obstructing fluid flow altogether. Current standards of care require 1 inch of catheter slack between the insertion site and the retainer system however, no retainer system can ensure such standards are adhered to and leaving the placement vulnerable to human error. Further, some retainer systems often include a rotatable coupling between the retainer and the anchor pad to allow for movement between the catheter and the patient during use. However, such retainer systems can often invert accidentally during placement resulting in catheter being coupled with the retainer incorrectly.

What is needed therefore is a retainer system that maintains correct slack profile of the catheter and mitigates incorrect assembly of the catheter and retainer system assembly.

### SUMMARY

In some aspects, the techniques described herein relate to a retainer system, including: a catheter having a bifurcation disposed at a proximal end of a catheter tube and a drainage extension leg and an inflation extension leg extending proximally from the bifurcation; an anchor pad defining a bottom surface and a top surface; and a retainer coupled to the top surface of the anchor pad and defining a first side and a second side, opposite the first side, the retainer including: a base; a cap hingedly coupled to the base; one or more extension leg sleeves defining a lumen and extending from the first side of the retainer; and a catheter tube sleeve defining a lumen and extending from the second side of the retainer.

In some aspects, the techniques described herein relate to a retainer system, wherein the retainer base defines a bifurcation channel configured to receive the bifurcation and communicates with the lumen of the catheter tube sleeve.

In some aspects, the techniques described herein relate to a retainer system, wherein the catheter tube sleeve lumen defines a non-linear path having one or more inflection points.

In some aspects, the techniques described herein relate to a retainer system, wherein the catheter tube sleeve lumen defines an inner diameter equal to or greater than an outer diameter of the catheter tube.

In some aspects, the techniques described herein relate to a retainer system, wherein the retainer is formed of a first material and one or both of the one or more extension leg sleeves and the catheter tube sleeve is formed of a second material, the first material defining relatively rigid material characteristics, the second material defining relatively more flexible material characteristics than the first material.

In some aspects, the techniques described herein relate to a retainer system, wherein the second material includes a relatively high friction coefficient and wherein an inner surface of the lumen of the catheter tube sleeve engages the catheter tube in a friction fit engagement.

In some aspects, the techniques described herein relate to a retainer system, wherein the inner surface of the lumen of the one or more extension leg sleeves engages a portion of an extension leg in a friction fit engagement.

In some aspects, the techniques described herein relate to a retainer system, wherein the catheter tube is formed of a third material that is relatively more flexible than the second material.

In some aspects, the techniques described herein relate to a retainer system, wherein a tip of the catheter tube sleeve extends a distance of 1 inch or greater from the second side of the retainer.

In some aspects, the techniques described herein relate to a retainer system, wherein the retainer is rotatably coupled to the top surface of the anchor pad.

In some aspects, the techniques described herein relate to a retainer system, wherein the anchor pad includes an adhesive layer disposed on the bottom surface and configured to secure the anchor pad to a skin surface of a patient.

In some aspects, the techniques described herein relate to a retainer system, wherein a distal tip of the catheter is configured to be disposed within a bladder of a patient and drain urine therefrom.

In some aspects, the techniques described herein relate to a method of retaining a catheter, including placing a portion of the catheter in a channel defined by a catheter tube sleeve extending from a base of a retainer; transitioning a cap of the retainer from an open position to a closed position; imparting a non-linear shape on the portion of the catheter disposed within the channel of the catheter tube sleeve; and securing the portion of the catheter within the catheter tube sleeve.

In some aspects, the techniques described herein relate to a method, wherein the non-linear shape includes a serpentine shape having one or more inflection points.

In some aspects, the techniques described herein relate to a method, wherein the portion of the catheter includes a proximal end of a catheter tube supported by a bifurcation, the bifurcation disposed within a channel of the base of the retainer, a portion of the bifurcation abuts against a surface of the base of the retainer.

In some aspects, the techniques described herein relate to a method, wherein the base further includes a first sleeve and second sleeve each extending from a first side of the base of the retainer, the catheter tube sleeve extending from a second side of the base, opposite the first side.

In some aspects, the techniques described herein relate to a method, wherein the catheter tube sleeve engages the portion of the catheter in a friction fit engagement.

In some aspects, the techniques described herein relate to a method, wherein the retainer is formed of a first material and the catheter tube sleeve is formed of a second material, the first material defining relatively rigid material characteristics, the second material defining relatively more flexible material characteristics than the first material.

In some aspects, the techniques described herein relate to a method, wherein the catheter is formed of a third material that is relatively more flexible than the second material.

In some aspects, the techniques described herein relate to a method, wherein a tip of the catheter tube sleeve extends a distance of 1 inch or greater from the base of the retainer.

In some aspects, the techniques described herein relate to a method, further including adhering a lower surface of an anchor pad to a skin surface of a patient, an upper surface of the anchor pad rotatably coupled to the base of the retainer.

In some aspects, the techniques described herein relate to a method, wherein a distal tip of the catheter is configured to be disposed within a bladder of a patient and drain urine therefrom.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of catheter retainer system in a closed position, in accordance with embodiments disclosed herein.

FIG. 2 shows a perspective view of catheter retainer system in an open position, in accordance with embodiments disclosed herein.

FIG. 3 shows a perspective view of catheter retainer system in a closed position, in accordance with embodiments disclosed herein.

FIG. 4 shows a perspective view of catheter retainer system in an open position, in accordance with embodiments disclosed herein.

FIGS. 5A-5B show close-up detail of a latch system of a retainer system, in accordance with embodiments disclosed herein.

FIGS. 5C-5D show close-up detail of a latch system of a retainer system, in accordance with embodiments disclosed herein.

FIG. 6 shows close-up detail of a catheter retainer system, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein. It is understood that the drawings are diagrammatic and schematic representations of exemplary embodiments of the invention and are neither limiting nor necessarily drawn to scale.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

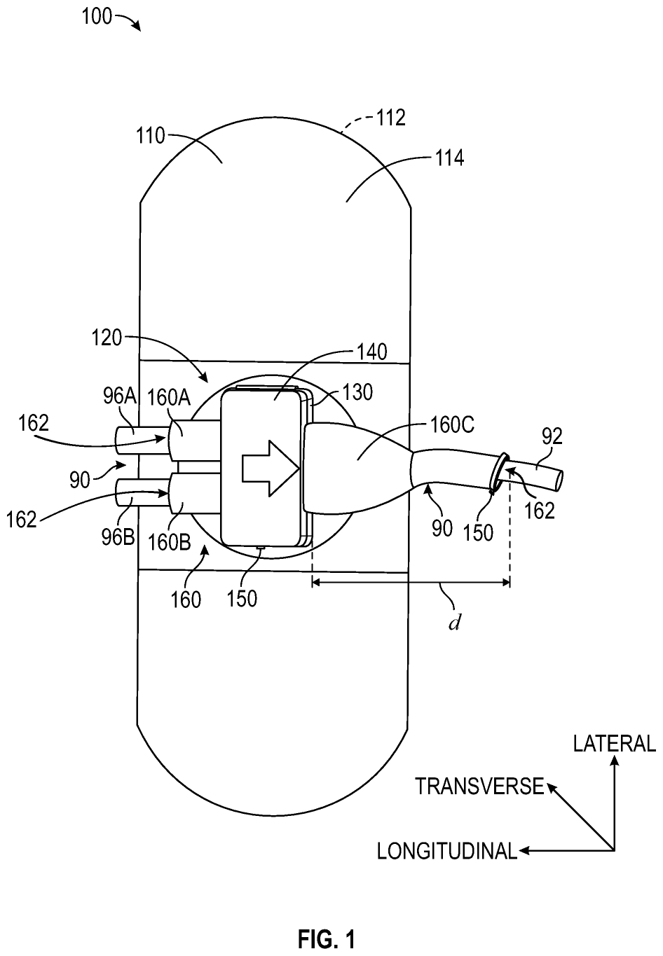

To assist in the description of embodiments described herein, as shown in FIG. 1, a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

FIG. 1 shows a perspective view of a retainer system (“system”) 100, generally including an anchor pad 110 and a retainer 120. The system 100 is configured to retain a portion of a catheter 90, the catheter 90 configured to drain a fluid from a cavity of a patient. Exemplary catheters 90 include fluid drainage catheters, urinary catheters, intermittent catheters, indwelling catheters, external catheters, Foley catheters, balloon catheters, intravascular catheters, peritoneal catheters, or the like. For example, a distal tip of the catheter 90 can be disposed within a bladder of a patient and configured to drain a fluid, e.g. urine therefrom. A proximal end of the catheter 90 is in fluid communication with a collection container, fluid bag, or similar receptacle.

The catheter 90 generally includes a flexible tubular body 92 defining one or more catheter lumen extending between a distal tip and a proximal end. In an embodiment, the proximal end of the catheter tube 92 is coupled to a bifurcation 94. The bifurcation 94 includes one or more extension legs extending proximally therefrom. A first extension leg 96A is in fluid communication with a first catheter lumen, e.g. a drainage lumen, a second extension leg 96B is in fluid communication with a second catheter lumen, e.g. an inflation lumen for a retention balloon. It will be appreciated that other numbers and configurations of extension legs and catheter lumen are also contemplate.

In an embodiment, the anchor pad 110 of the retainer system 100 is configured to secure the retainer 120 to a patient's skin. The anchor pad 110 includes adhesive layer 112 disposed on a lower surface thereof and configured to adhere to the skin of a patient. The retainer 120 is coupled to an upper surface 114 of the anchor pad 110. In an embodiment, the retainer 120 is rotatably coupled to the anchor pad to facilitate aligning the catheter 90 to facilitate fluid flow, and to prevent kinking or collapse of the catheter tube 90. The anchor pad 110 is formed of a woven or non-woven flexible polymer material, or polymer foam material, or similar suitable material. Optionally, the anchor pad further includes one or more release layers disposed on the adhesive layer 112 and configured to protect the adhesive layer 112 during transport and storage.

In an embodiment, the retainer 120 is configured to retain a portion of the catheter 90, i.e. a portion of one or more of the catheter tube 92, bifurcation 94, and one or more extension legs, e.g. a portion of the first, drainage extension leg 96A and a portion of the second inflation extension leg 96B. The retainer 120 is configured to receive a portion of the catheter 90 in a first (“correct”) orientation but to prevent receiving a portion of the catheter 90 in a second orientation, different from the first orientation. Advantageously, the retainer 120 mitigates incorrect assembly of the catheter 90 and the retainer 120, i.e. mitigates longitudinal inversion of the catheter 90 where a distal end of the catheter 90 is incorrectly aligned with a proximal end of the retainer 120, or mitigates lateral inversion of the catheter 90 where a drainage extension leg 96A is aligned with a channel of the retainer 120 that is meant to receive the inflation extension leg 96B and visa versa.

The retainer 120 generally includes a base 130 coupled to the upper surface 114 of the anchor pad 110 and defines one or more channels 132 extending between a first side 122 and a second side 124 of the retainer 120. In an embodiment, the one or more channels includes a first channel 132A configured to receive a portion of the first extension leg 96A, a second channel 132B configured to receive a portion of the second extension leg 96B, and a third channel 132C configured to receive a portion of the bifurcation 94 and/or a portion of the catheter tube 92. In an embodiment, the one or more channels 132 are aligned asymmetrically relative to a central longitudinal axis of the retainer 120 so as to mitigate incorrect assembly of the catheter 90 and the retainer 120, as described herein.

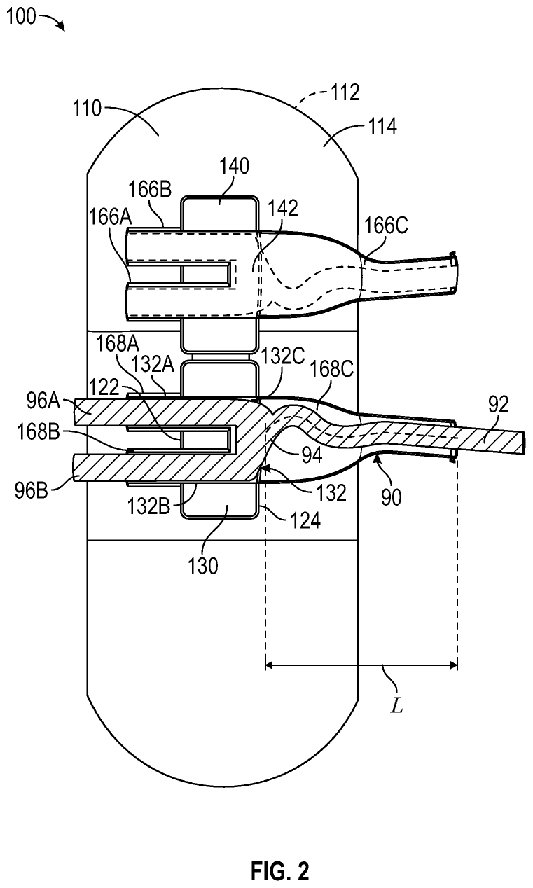

For example, as shown in FIG. 2, the first channel 132A for receiving the drainage extension leg 96A, and the third channel 132C for receiving the bifurcation 94 and/or catheter tube 92 can be substantially aligned along a longitudinal axis and the second channel 132B for receiving the inflation extension leg 96B can extend at an angle thereto. However, it will be appreciated that other configurations of channels 132A, 132B, 132C are contemplated to fall within the scope of the present invention.

In an embodiment, the retainer 120 further includes a cap 140 hingedly coupled with the base 130 and transitionable between and open position (FIG. 2) and a closed position (FIG. 1). As shown in FIG. 1, in the closed position, the cap 140 extends across a top surface of the base 130 and retains the catheter 90, or portions thereof, within the channels 132 of the base 130. As shown in FIG. 2, in the open position, the cap 140 pivots away from the base 130 to allow for ingress or egress of the catheter 90 to/from the base 130. The cap 140 is coupled to the base 130 by a living hinge, mechanical hinge, or the like.

In an embodiment, the cap 140 includes one or more cap channels 142 that align with the one or more channels 132 of the base 130. The one or more cap channels 142 co-operate with the one or more base channels 132 to encircle the portions of the catheter 90 disposed therein. In an embodiment, the cap 140 is releasably secured in the closed position by a latch mechanism 150 as described in more detail herein. In an embodiment, the retainer 120 is formed from a first material that displays resilient or substantially rigid mechanical properties. Exemplary materials include plastic, polymer, metal, alloy, composites, polypropylene, or similar suitable materials.

In an embodiment, the retainer 120 includes one or more sleeves 160 extending along a longitudinal axis from one or both of the first side 122 and the second side 124 of the retainer 120. The sleeves 160 are formed of a second material, different from the first material of the retainer 120. The second material is a relatively softer, more pliable or more elastic material relative to the first material of the retainer 120. In an embodiment, the second material can have a relatively higher friction coefficient relative to the first material. Advantageously, the second material can facilitate gripping the portion of the catheter 90 disposed therein and still allow a elastic movement of a portion of the catheter 90 disposed within the sleeve 160 relative to the retainer 120. Exemplary materials of the second material include plastics, polymers, elastomers, rubber, silicone rubber, or the like.

In an embodiment, the second material of the sleeve 160 displays different mechanical characteristics from a third material used to form the catheter 90, or portions thereof. The third material of the catheter 90 is relatively more flexible than the mechanical characteristics of the second material. As such, while both the sleeve 160 and the catheter 90 are flexible, the catheter 90 is more flexible than the sleeve and as such, the shape of the sleeve lumen 162 imparts a similar shape on the portion of the catheter 90 disposed therein. In an embodiment, an inner diameter of the sleeve lumen 162 is equal to or slightly larger than an outer diameter of the catheter 90. As such, the catheter 90, or portion thereof fits snugly within the catheter tube sleeve lumen 162.

In an embodiment, the sleeve 160 defines a sleeve lumen 162 that aligns with a channel 132 of the retainer 120. For example, a first sleeve 160A extends from a first side 122 of the retainer 120. The first sleeve 160A defines a first sleeve lumen 162A that axially aligns with a first channel 132A of the retainer 120. The first sleeve 160A is configured to encircle a second portion of the drainage extension leg 96A that extends from the first channel 132A of the retainer 120. Further, a second sleeve 160B also extends from a first side 122 of the retainer 120. The second sleeve 160B defines a second sleeve lumen 162B that axially aligns with a second channel 132B of the retainer 120. The second sleeve 160B is configured to encircle a second portion of the inflation extension leg 96B that extends from the second channel 132B of the retainer 120.

In an embodiment, a third sleeve 160C, or catheter tube sleeve 160C, extends from a second side 124 of the retainer 120. The catheter tube sleeve 160C defines a third sleeve lumen 162C that axially aligns with the third channel 132C. The catheter tube sleeve 160C is configured to encircle a portion of the bifurcation 94 and/or the catheter tube 92 extending from the retainer 120. In an embodiment, an axis of one or more of the first sleeve lumen 162A, second sleeve lumen 162B, and third sleeve lumen 162C extends linearly from the retainer 120, e.g., along a longitudinal axis.

In an embodiment, an axis of one or more of the first sleeve lumen 162A, second sleeve lumen 162B, and third sleeve lumen 162C extends along a non-linear path from the retainer 120 and includes one or more inflection points. As such, as shown in FIG. 2 for example, the axis of the sleeve lumen defines a serpentine, or sigma, shape.

As shown in FIGS. 1-2, a linear distance (d) extends between the second side 124 of the retainer 120 and an outer edge of the sleeve 160, e.g., an outer edge of the catheter tube sleeve 160C. Due to the non-linear shape of the sleeve lumen 162, a length (L) of catheter 90 disposed within the catheter tube sleeve 160C is greater than the distance (d). In an embodiment, the ratio of distance (d) to length (L) is greater than 1:1. In an embodiment, exemplary ratios of distance (d) to length (L) includes between 1:1.1 and 1:2. However, greater or lesser ratios are also contemplated. In an embodiment, the distance (d) is equal to or greater than 1 inch, as such, the amount of slack, i.e., length (L), imparted on the catheter 90 by the retainer system 100 is greater than 1 inch.

As noted, the sleeve 160 imparts the shape of the sleeve lumen 162 onto the portion of the catheter 90 disposed therein. As such, the sleeve 160, e.g., catheter tube sleeve 160C maintains a predetermined amount of slack in the catheter 90 between the retainer 120 and the distal tip of the catheter 90. Advantageously, the predetermined amount of slack, the flexibility of the sleeve 160 and the flexibility of the catheter 90 allows serpentine shape to elastically deform to a relatively more linear shape during use and mitigates any dislodgement of the distal tip of the catheter disposed within the body of the patient.

Advantageously, the serpentine shape mitigates human error in the placement of the retainer system 100 mitigating either too little slack since sufficient slack is already gathered when placing the catheter 90 in the retainer 120. Further, the system 100 mitigates problems associated with too much slack since the sleeve 160 provides additional support and strain relief to the portion of the catheter 90 extending from the retainer 120, mitigating kinking or collapse. Advantageously, the non-linear path of the sleeve lumen can grip the portion of the catheter 90, disposed therein, and mitigate longitudinal movement of the catheter 90 through the retainer 120. Advantageously, the one or more sleeves 160 can prevent kinking or collapse of the catheter 90 within 1 inch of the retainer 120.

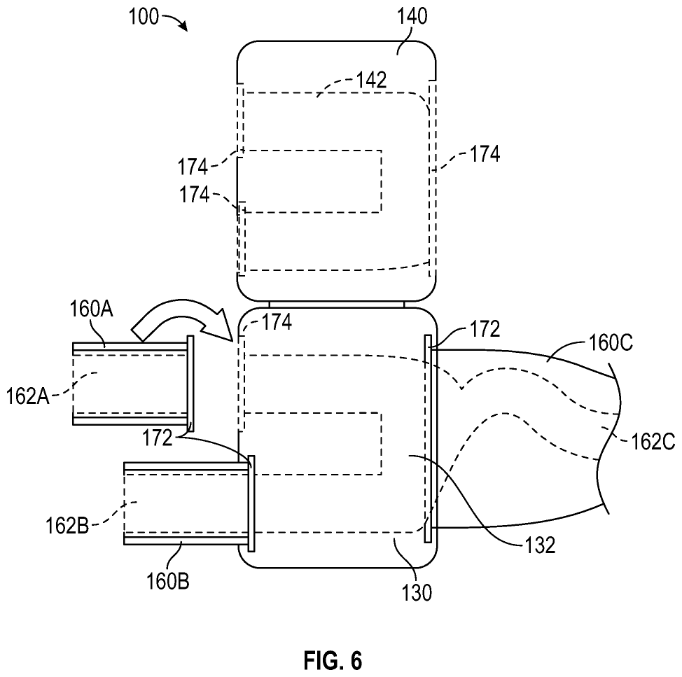

In an embodiment, as shown in FIG. 6, one or more of the sleeves 160 includes a lip 172 extending radially about one end of the sleeve. The lip 172 is configured to engage a groove 174 extending about an opening to a channel 132. In an embodiment, the sleeve 160 is slidably engaged with a portion of one of the drainage extension leg 96A, the inflation extension leg 96B, or the catheter tube 92, and with the lip 172 disposed adjacent the retainer 120. With a portion of the bifurcation 94 engaged with the retainer base, the lip 172 engages the groove 174 disposed in the base 130. As the cap 140 closes over the base 130, a groove 174 disposed in the cap closes over the lip 172 of the sleeve retaining the sleeve 160 engaged with the retainer 120.

In an embodiment, a top half 166 of the sleeve 160 is coupled with the cap 140 and a bottom half 168 of the sleeve 160 is coupled with base 130. The top half 166 is aligned with the cap channel 142 and the bottom half is aligned with the base channel 132. As the cap 140 transitions to the closed position, the top half 166 co-operates with the bottom half 168 to define the sleeve 160 and enclose the portion of the catheter 90 therein. For example, a first top half 166A co-operates with a first bottom half 168A to define a first sleeve 160A. Similarly, a second top half 166B co-operates with a second bottom half 168B to define a second sleeve 160B, and a third top half 166C co-operates with a third bottom half 168C to define a third sleeve 160B. In an embodiment, one or more of the sleeves 160 includes one or more latching mechanisms 150, as described herein, and configured to secure the top half 166 of the sleeve 160 with the bottom half 168 of the sleeve 160.

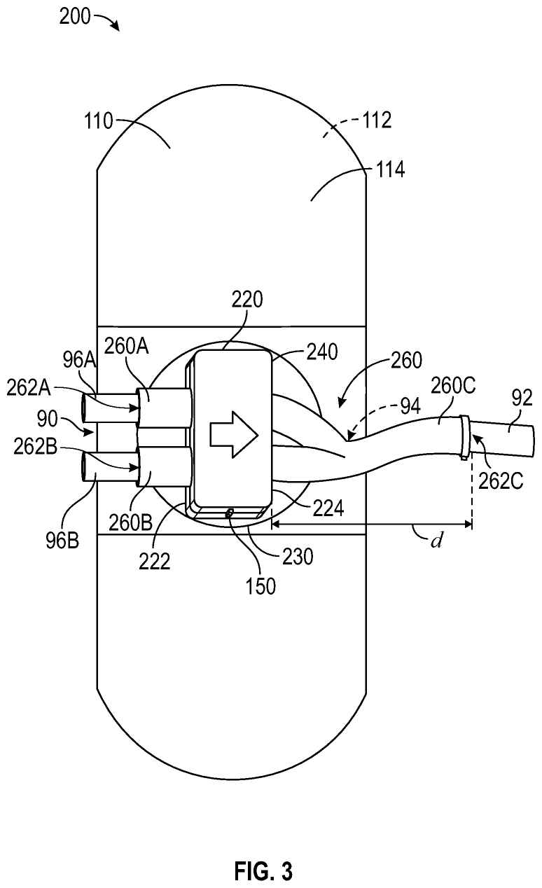

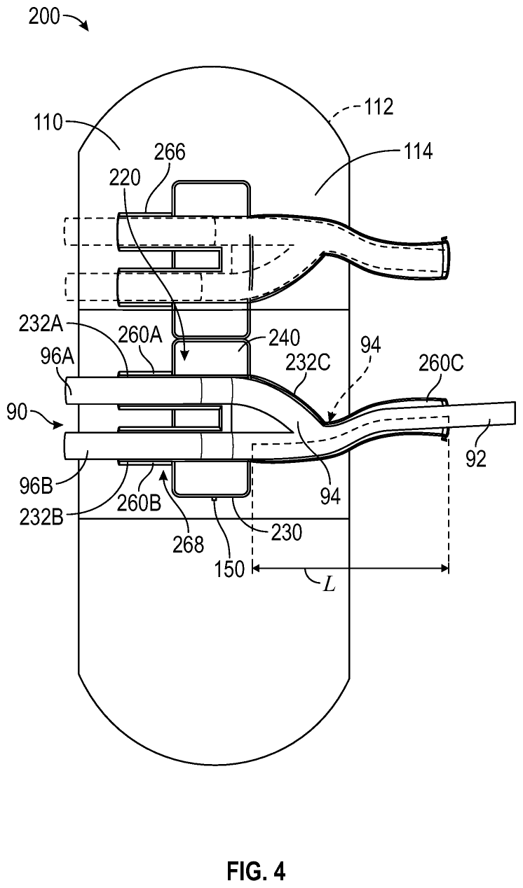

FIGS. 3-4 show an embodiment of a catheter retainer system 200 generally including an anchor pad 110, as described herein, and a retainer 220. The retainer 220 includes a base 230 coupled to the anchor pad 110 and a cap 240 hingedly coupled the base. The retainer 220 further includes a latch 150, as described herein, to releasably secure the cap 240 to the base 230.

In an embodiment, the base 230 of the retainer 220 defines a first channel 232A for retaining a portion of a first or drainage extension leg 96A and a second channel 232B for retaining a portion of a second or inflation extension leg 96B.

A first sleeve 260A extends from a first side 222 of the retainer 220 and defines a first sleeve lumen 262A that aligns with the first channel 232A and encircles a portion of the drainage extension leg 96A extending from the retainer 220. A second sleeve 260B extends from the first side 222 of the retainer 220 and defines a second sleeve lumen 262B that aligns with a second channel 232B and encircles a portion of the inflation extension leg 96B extending from the retainer 220.

A third sleeve 260C extends from a second side 224 of the retainer 220 and defines a “Y-shaped” third channel 232C configured to retain the bifurcation 94 of the catheter 90 and a portion of one or more of the drainage extension leg 96A, the inflation extension leg 96B, and the catheter tube 90.

As shown in FIGS. 3-4, an outer-most edge of the third sleeve 260C extends to a distance (d) and defines a non-linear central axis to a lumen of the third sleeve 260C. As described herein, the length (L) of the lumen is greater than the distance (d). Worded differently, a ratio of the distance (d) to lumen length (L) is greater than 1:1, for example between 1:1.1 and 1:2. In an embodiment, the distance (d) is equal to or greater than 1 inch and as such the lumen length (L) is greater than 1 inch. Advantageously, the non-linear lumen length (L) imparts a predetermined slack on the catheter 90 disposed therein which mitigates dislodging the distal tip of the catheter 90.

In an embodiment, one or more sleeves 260 includes a lip 172 disposed at one and extending radially therefrom and configured to engage a groove disposed in the retainer 220. In the closed position, the lip 172 is secured within the groove 174 and retains the sleeve with the retainer 220.

In an embodiment, the one or more sleeves 260 include a top half 266 and a bottom half 268 that co-operate to define the sleeve 260, as described herein. The top half 266 is coupled with the cap 240 and the bottom half 268 is coupled with the base 230. In the closed position, the top half 266 is secured to the bottom half 268 with one or more latch mechanisms 150 as described herein. Advantageously, the bifurcation 94 abuts against a surface of the sleeve 260C to mitigate axial or rotational movement of the catheter 90 relative to the retainer system 200.

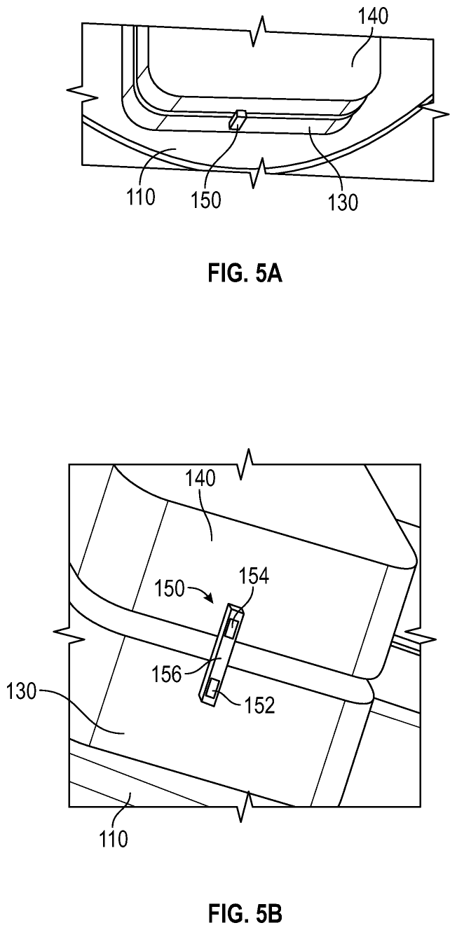

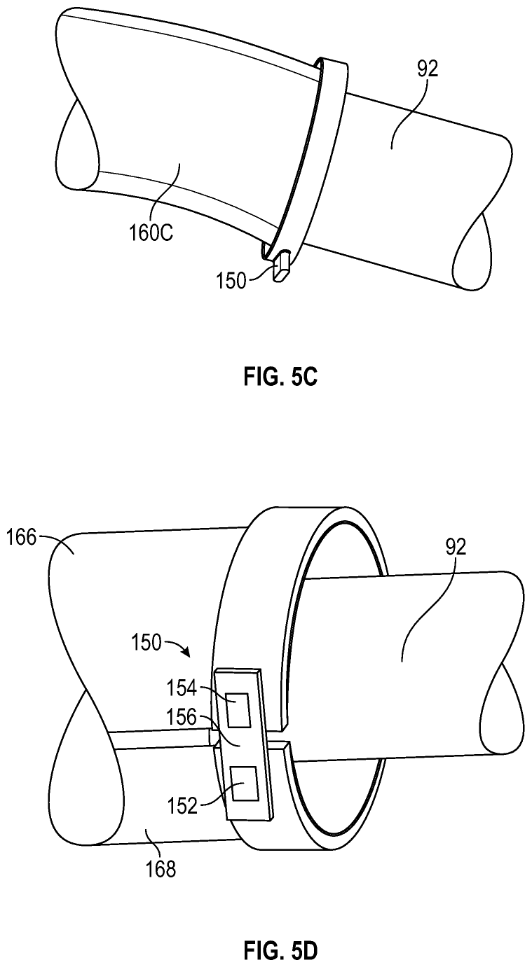

FIGS. 5A-5D show further details of an exemplary latching mechanism (“latch”) 150. In an embodiment, the latch 150 includes a first pawl 152 extending from a base 130 and a second pawl 154 extending from a cap 140. One or both of the first pawl 152 and the second pawl 154 includes a hook or barb. The latch 150 further includes a strap 156 including a first aperture configured to engage the first pawl 152 and a second aperture configured to engage the second pawl 154. The strap 156 is formed from a flexible material configured to engage each of the first pawl 152 and the second pawl 154 in a friction fit, snap-fit, or press-fit engagement. Further the hook or barb can further secure the strap 156 to one of the first pawl 152 and the second pawl 154.

In an exemplary method of use, a retainer system, e.g., retainer system 100 or retainer system 200, is provided as disclosed herein. The retainer system generally includes a anchor pad 110 and a retainer, e.g., retainer 120 or retainer 220, and is configured to releasably secure a catheter 90 to a skin surface of a patient.

Initially, a distal tip of a catheter tube 92 is placed within a cavity of a patient, e.g. a bladder, and is configured to drain a fluid therefrom. A proximal end of the catheter tube 92 includes a bifurcation 94 and one or more extension legs 96 extending proximally therefrom. For example, a drainage extension leg 96A is in fluid communication with a drainage lumen of catheter tube 92 and provides fluid communication between the distal tip of the catheter tube 92 and a collection bag, optionally by way of a drainage tube. Further, an inflation extension leg is in fluid communication with an inflation lumen of the catheter tube 92 and provides fluid communication with a retention balloon disposed adjacent the distal tip of the catheter tube 92 and configured to retain the distal tip within the cavity. The inflation extension leg is in fluid communication with a source of inflation fluid.

The retainer 120, 220 is transitioned to the open position and a portion of the catheter 90 is disposed within a channel of the retainer base 130. In an embodiment, one or more of a portion of the drainage extension leg, the inflation extension leg, the bifurcation 94, and the catheter tube 92 is retained within the channel of the base 130, 230.

In an embodiment, a portion of the catheter tube 92 is disposed within a lumen 162C of the catheter tube sleeve 160C that extends from the second side 124 of the retainer 120.

In an embodiment, a portion of the drainage extension leg 96A is disposed within a lumen 162A of a first extension leg sleeve 160A that extends from the first side 122 of the retainer 120, and a portion of the inflation extension leg 96B is disposed within a lumen 162B of a second extension leg sleeve 160B that extends from the first side 122 of the retainer 120.

The lumen 162C of the catheter tube sleeve 160C defines a non-linear shape, for example a serpentine shape including one or more inflection points. The material of the catheter tube sleeve 160C is flexible but defines a relatively greater rigidity than that of the material of the catheter tube 92. As such, the catheter tube sleeve 160C imparts a non-linear, serpentine shape on the portion of the catheter tube 92 disposed therein. One or more of the friction coefficient of the material of the catheter tube sleeve 160C, and the one or more inflection points mitigates longitudinal movement of the catheter 90 relative to the retainer 120. The non-linear shape of the catheter tube sleeve lumen 162C, 262C, imparts a predetermined amount of slack on the catheter 90 that allows for movement between the distal tip of the catheter 90 and the retainer 120, 220, mitigating dislodgement of the catheter tip and discomfort to the patient during use. Further, a portion of the bifurcation 94 abuts against a portion of the retainer 120 to mitigate longitudinal movement of the catheter 90 relative to the retainer 120.

In an embodiment, as shown in FIGS. 3-4, a portion of the bifurcation 94 is retained within the catheter tube sleeve 260C. As such, a portion of the bifurcation 94 abuts against a portion of the catheter tube sleeve 260C to mitigate longitudinal movement of the catheter 90 relative to the retainer 220. Further the non-linear shape of the catheter tube sleeve 260C imparts a non-linear shape on the portion of the catheter tube 92 disposed therein and mitigates longitudinal movement of the catheter 90 relative to the retainer 220 and imparts a predetermined amount of slack on the catheter 90.

With the catheter 90 retained within the base 130, the cap 140 is pivoted from the open position to the closed position that secures the catheter 90 within the retainer. In an embodiment, a top half of one of one or more of the first sleeve, second sleeve, and catheter tube sleeve aligns with a bottom half of the respective the first sleeve, second sleeve, and catheter tube sleeve to secure one or more of the drainage extension leg, inflation extension leg, bifurcation, and catheter tube therein.

In an embodiment, a latch 150 releasably secures the cap 140 to the base 130 in the closed position. In an embodiment, a latch 150 secures a top half 166 of one or more of the first sleeve, second sleeve, and catheter tube sleeve to a bottom half 168 of a respective first sleeve, second sleeve, and catheter tube sleeve.

Advantageously the retainer 120, 220 is rotatable relative to the anchor pad 110 to align the drainage tube with collection container and facilitate gravitational induced fluid flow therethrough. During use, the retainer can pivot causing the catheter tube 92 to kink or collapse. For example, the retainer can pivot through more than 90°, through more than 180°, or through more than 360° although greater or lesser angles are also contemplated. As such the catheter tube 92 can bend excessively causing kinking or collapse. Advantageously, one or more of the first sleeve, second sleeve, and catheter tube sleeve and support the portion of the catheter 90 disposed therein and mitigate kinking or collapse. In an embodiment, one or more of the first sleeve, second sleeve, and catheter tube sleeve extends more than 1 inch from the retainer 120 to mitigate kinking or collapse of the catheter 90.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A retainer system, comprising:
a catheter having a bifurcation disposed at a proximal end of a catheter tube and a drainage extension leg and an inflation extension leg extending proximally from the bifurcation;
an anchor pad defining a bottom surface and a top surface; and
a retainer coupled to the top surface of the anchor pad and defining a first side and a second side, opposite the first side, the retainer comprising:
a base;
a cap hingedly coupled to the base;
one or more extension leg sleeves defining a lumen and extending from the first side of the retainer; and
a catheter tube sleeve defining a lumen and extending from the second side of the retainer.

2. The retainer system according to claim 1, wherein the retainer base defines a bifurcation channel configured to receive the bifurcation and communicates with the lumen of the catheter tube sleeve.

3. The retainer system according to claim 1, wherein the catheter tube sleeve lumen defines a non-linear path having one or more inflection points.

4. The retainer system according to claim 1, wherein the catheter tube sleeve lumen defines an inner diameter equal to or greater than an outer diameter of the catheter tube.

5. The retainer system according to claim 1, wherein the retainer is formed of a first material and one or both of the one or more extension leg sleeves and the catheter tube sleeve is formed of a second material, the first material defining relatively rigid material characteristics, the second material defining relatively more flexible material characteristics than the first material.

6. The retainer system according to claim 5, wherein the second material includes a relatively high friction coefficient and wherein an inner surface of the lumen of the catheter tube sleeve engages the catheter tube in a friction fit engagement.

7. The retainer system according to claim 6, wherein the inner surface of the lumen of the one or more extension leg sleeves engages a portion of an extension leg in a friction fit engagement.

8. The retainer system according to claim 5, wherein the catheter tube is formed of a third material that is relatively more flexible than the second material.

9. The retainer system according to claim 1, wherein a tip of the catheter tube sleeve extends a distance of 1 inch or greater from the second side of the retainer.

10. The retainer system according to claim 1, wherein the retainer is rotatably coupled to the top surface of the anchor pad.

11. The retainer system according to claim 1, wherein the anchor pad includes an adhesive layer disposed on the bottom surface and configured to secure the anchor pad to a skin surface of a patient.

12. The retainer system according to claim 1, wherein a distal tip of the catheter is configured to be disposed within a bladder of a patient and drain urine therefrom.

13. A method of retaining a catheter, comprising:
placing a portion of the catheter in a channel defined by a catheter tube sleeve extending from a base of a retainer;
transitioning a cap of the retainer from an open position to a closed position;
imparting a non-linear shape on the portion of the catheter disposed within the channel of the catheter tube sleeve; and
securing the portion of the catheter within the catheter tube sleeve.

14. The method according to claim 13, wherein the non-linear shape includes a serpentine shape having one or more inflection points.

15. The method according to claim 13, wherein the portion of the catheter includes a proximal end of a catheter tube supported by a bifurcation, the bifurcation disposed within a channel of the base of the retainer, a portion of the bifurcation abuts against a surface of the base of the retainer.

16. The method according to claim 13, wherein the base further includes a first sleeve and second sleeve each extending from a first side of the base of the retainer, the catheter tube sleeve extending from a second side of the base, opposite the first side.

17. The method according to claim 13, wherein the catheter tube sleeve engages the portion of the catheter in a friction fit engagement.

18. The method according to claim 13, wherein the retainer is formed of a first material and the catheter tube sleeve is formed of a second material, the first material defining relatively rigid material characteristics, the second material defining relatively more flexible material characteristics than the first material.

19. The method according to claim 18, wherein the catheter is formed of a third material that is relatively more flexible than the second material.

20. The method according to claim 13, wherein a tip of the catheter tube sleeve extends a distance of 1 inch or greater from the base of the retainer.

21. The method according to claim 13, further including adhering a lower surface of an anchor pad to a skin surface of a patient, an upper surface of the anchor pad rotatably coupled to the base of the retainer.

22. The method according to claim 13, wherein a distal tip of the catheter is configured to be disposed within a bladder of a patient and drain urine therefrom.
